(19) United States
(12) Patent Application Publication
(10) Pub. No.: US 2007/0093869 A1
(43) Pub. Date: Apr. 26, 2007

(54) Device, system, and method for contracting tissue in a mammalian body

(75) Inventors: Eliot Bloom, Hopkinton, NH (US); Nareak Douk, Lowell, MA (US); Nasser Rafiee, Andover, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: MEDTRONIC VASCULAR, INC., SANTA ROSA, CA (US)

(21) Appl. No.: 10/560,986

(22) PCT Filed: Jun. 21, 2004
(86) PCT No.: PCT/US04/19829

§ 371 Date: Dec. 15, 2005

Related U.S. Application Data
(60) Provisional application No. 60/480,473, filed on Jun. 20, 2003.

(51) Int. Cl. A61B 17/08

(52) U.S. Cl. 606/219

(57) ABSTRACT

A device, a system and a method for contracting tissue in a mammalian body. The contracting device comprises a body and a plurality of legs radially splayed there from. Each leg includes a snap-acting spring tip. When a force is applied to the device, the tips operate to transform the device from a deployment state into a treatment state. Each leg may also be bent at one or more deformation elements in response to the application of force. The system comprises a contracting device slidably received within a delivery catheter. The method of contracting tissue in a mammalian body comprises delivering a contracting device in a lumen of a catheter proximate a treatment area, releasing the contracting device from the catheter, positioning legs of the contracting device on tissue to be contracted, exerting a force on the contracting device, transforming the device into a treatment state, and reducing a compass of the tissue in response to the attainment of the treatment state.

### PRIORITY CLAIM

This application claims the benefit of U.S. Provisional Application No. 60/480,473, Titled “Method and System for Reducing Mitral Valve Regurgitation” by Eliot Bloom, et al., filed Jun. 20, 2003, which is hereby incorporated by reference.

### TECHNICAL FIELD

This invention relates generally to medical devices and particularly to a device, system, and method for contracting tissue in a mammalian body.

### BACKGROUND OF THE INVENTION

The heart is a four-chambered pump that moves blood efficiently through the vascular system. Blood enters the heart through the vena cava and flows into the right atrium. From the right atrium, blood flows through the tricuspid valve and into the right ventricle, which then contracts and forces blood through the pulmonic valve and into the lungs. Oxygenated blood returns from the lungs and enters the heart through the left atrium and passes through the bicuspid mitral valve into the left ventricle. The left ventricle contracts and pumps blood through the aortic valve into the aorta and to the vascular system.

The mitral valve consists of two leaflets (anterior and posterior) attached to a fibrous ring or annulus. In a healthy heart, the mitral valve leaflets overlap during contraction of the left ventricle and prevent blood from flowing back into the left atrium. However, due to various cardiac diseases, the mitral valve annulus may become distended, causing the leaflets to remain partially open during ventricular contraction and thus allowing regurgitation of blood into the left atrium. This results in reduced ejection volume from the left ventricle, causing the left ventricle to compensate with a larger stroke volume. The increased workload eventually results in dilation and hypertrophy of the left ventricle, further enlarging and distorting the shape of the mitral valve. If left untreated, the condition may result in cardiac insufficiency, ventricular failure, and death.

It is common medical practice to treat mitral valve regurgitation by valve replacement or repair. Valve replacement involves an open-heart surgical procedure in which the patient's mitral valve is removed and replaced with an artificial valve. This is a complex, invasive surgical procedure with the potential for many complications and a long recovery period.

Mitral valve repair includes a variety of procedures to reshape or reposition the leaflets to improve closure of the valve during ventricular contraction. Correction of the regurgitation may not require repair of the valve leaflets themselves, but simply a reduction in the size of the mitral valve annulus, which can become distended. A common repair procedure involves implanting an annuloplasty ring on the mitral valve annulus. The annuloplasty ring generally has a smaller diameter than the distended annulus, and when sutured to the annulus, the annuloplasty ring draws the annulus into a smaller configuration, bringing the mitral valve leaflets closer together and providing improved closure during ventricular contraction.

Annuloplasty rings may be rigid, flexible, or have both rigid and flexible segments. Rigid annuloplasty rings have the disadvantage of causing the mitral valve annulus to be rigid and unable to flex in response to the contractions of the ventricle, thus inhibiting the normal movement of the mitral valve that is required for it to function optimally. Flexible annuloplasty rings are frequently made of Dacron® fabric and must be sewn to the annular ring with a line of sutures. Scar tissue formation from the multiple stitches may lead to loss of flexibility and function of the mitral valve. Similarly, combination rings must generally be sutured in place and also cause scar tissue formation and loss of mitral valve flexibility and function.

Annuloplasty rings have been developed that do not require suturing. U.S. Pat. No. 6,565,603 discloses a combination rigid and flexible annuloplasty ring that is inserted into the fat pad of the atrioventricular groove, which surrounds the mitral valve annulus. Although this device avoids the need for sutures, it must be placed within the atrioventricular groove with great care to prevent tissue damage to the heart.

Therefore, it would be desirable to provide a device, system, and method for treating mitral valve regurgitation that overcome the aforementioned and other disadvantages.

### SUMMARY OF THE INVENTION

One aspect of the present invention is a device for contracting tissue in a mammalian body, comprising a body and a plurality of legs radially splayed from the body. Each leg includes a snap-acting spring tip for piercing engagement with the tissue. A sufficient axial force will transform the device from a deployed state to a treatment state under the action of the snap-acting spring tips. The treatment state will apply contraction force to the tissue engaged by the tips. Each leg may further include at least one deformation element that plastically bends in response to application of the sufficient force.

Another aspect of the present invention is a system for contracting tissue in a mammalian body that includes the above-described contracting device and further comprises a delivery catheter. The contracting device is elastically collapsible to be slidably received within a lumen of the delivery catheter.

Yet, another aspect of the present invention is a method of contracting tissue in a mammalian body. A contracting device is delivered in a lumen of a catheter proximate a treatment area. The contracting device is released from the catheter. Legs of the contracting device are positioned on tissue to be contracted. An axial force is exerted on the contracting device. The force is redirected. A compass of the tissue is reduced in response to the redirection of the force.

The aforementioned and other features and advantages of the invention will become further apparent from the following detailed description of the presently preferred embodiments, read in conjunction with the accompanying drawings, which are not to scale. The detailed description and drawings are merely illustrative of the invention rather than limiting, the scope of the invention being defined by the appended claims and equivalents thereof.

### BRIEF DESCRIPTION OF THE DRAWINGS

FIG. 1 is an isometric view of one embodiment of a device for contracting tissue in a mammalian body in accordance with the present invention, the device being shown in its deployed state;

FIG. 2 is an isometric view of the device of FIG. 1 shown in its treatment state;

FIGS. 3 and 4 are illustrations of the snap-acting spring tip of the device of FIG. 1, showing the tip at two stages of manufacture;

FIGS. 5 and 6 are illustrations of alternative deformation elements for a leg of the device of FIG. 1, in accordance with the present invention;

FIGS. 7 and 8 are longitudinal cross-sectional views of one embodiment of a system for contracting tissue in a mammalian body in accordance with the present invention, shown at two stages of deployment of the contracting device of FIG. 1, the contracting device being shown in toto and a guiding catheter being shown in cross section;

FIGS. 9-13 are views showing a progression of placement of a contracting device proximate a mitral valve, in accordance with the present invention; and

FIG. 14 is a flow diagram of one embodiment of a method of contracting tissue in a mammalian body, in accordance with the present invention.

The same reference numbers are used throughout the drawings to refer to the same parts.

### DETAILED DESCRIPTION OF THE PRESENTLY PREFERRED EMBODIMENTS

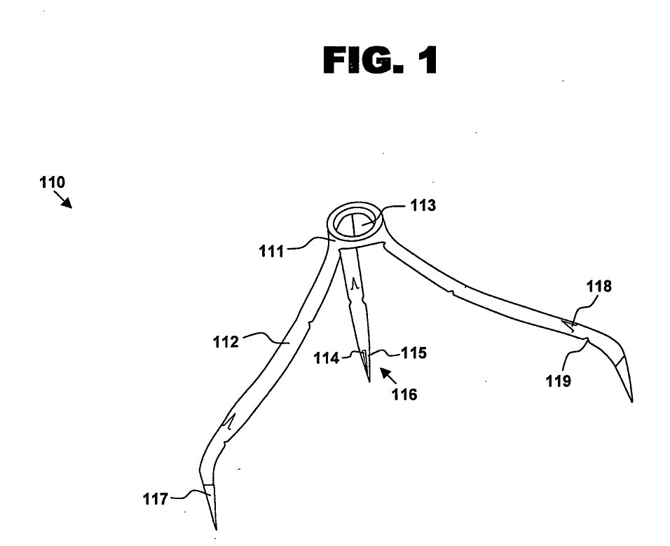

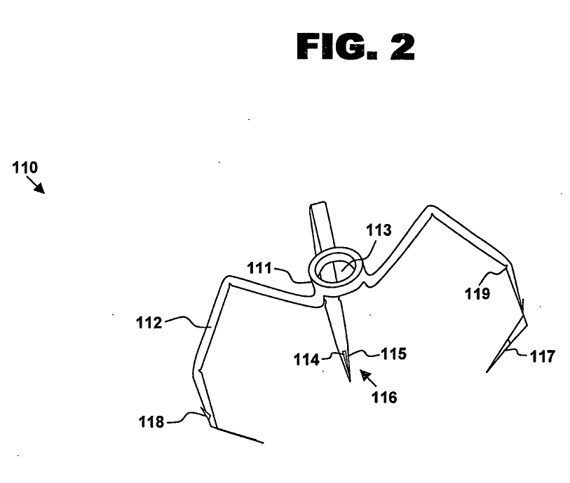

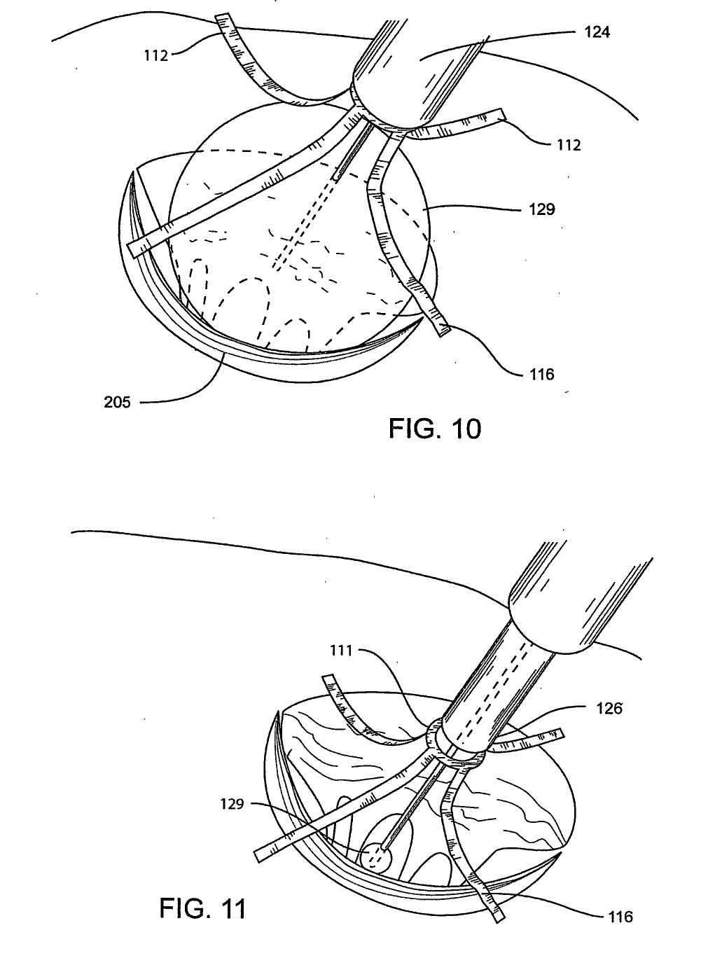

One aspect of the present invention is a device for contracting tissue in a mammalian body. One embodiment of the device, in accordance with the present invention, is illustrated in FIGS. 1 and 2 at 110. As illustrated, contracting device 110 comprises body 111 having a longitudinal axis. In the current example, three legs 112 extend from body 111 and are radially splayed about the longitudinal axis. One skilled in the art will appreciate that legs 112 may be varied in number, length, and spacing around body 111. For example, FIGS. 10-13 illustrate a four-legged embodiment of contracting device 110.

Contracting device 110 is designed to be positioned using intravascular catheterization techniques. Alternatively, surgical or minimally invasive, i.e. endoscopic techniques may be used to place contracting device 110. Although described below in the context of treating mitral valve regurgitation by radially contracting the valve annulus, the contracting device of the invention may also be deployed at other locations in the body and may be used to reduce the compass of other openings or the transverse dimensions of other structures within the body.

In the present embodiment, contracting device 110 may be fabricated from a section of tubing having evenly spaced longitudinal slots cut from one end of the tubing to form generally annular body 111 and flexible legs 112. The slots may be, for example, rectangular, u-shaped, v-shaped, or Ω-shaped (omega-shaped). A central lumen of the tubing forms aperture 113 in body 111. In another embodiment, contracting device 110 may be manufactured by cutting, stamping, or otherwise forming the device from flat sheets or other material not previously shaped into a tube. In any embodiment of the invention, legs 112 may be formed separately from body 111 and subsequently attached thereto, thus assembling a whole contracting device 110.

Contracting device 110 is manufactured using one or more biocompatible materials. At least legs 112 of contracting device 110 comprise a biocompatible material capable of being preset into a desired shape, for example that shown in FIG. 1. Such materials include, but are not limited to, a nickel-titanium alloy, a nickel-cobalt alloy, another cobalt alloy, a thermoset plastic, a stainless steel alloy, a suitable biocompatible shape-memory material, a suitable biocompatible superelastic material, combinations thereof, and the like. An antithrombotic component may be included in the chemical composition of a polymer used to form the device. Optionally, a polymeric or metallic device may be coated with a polymer that releases an anticoagulant and thereby reduces the risk of thrombus formation. If desired, additional therapeutic agents or combinations of agents may be used, including antibiotics and anti-inflammatories.

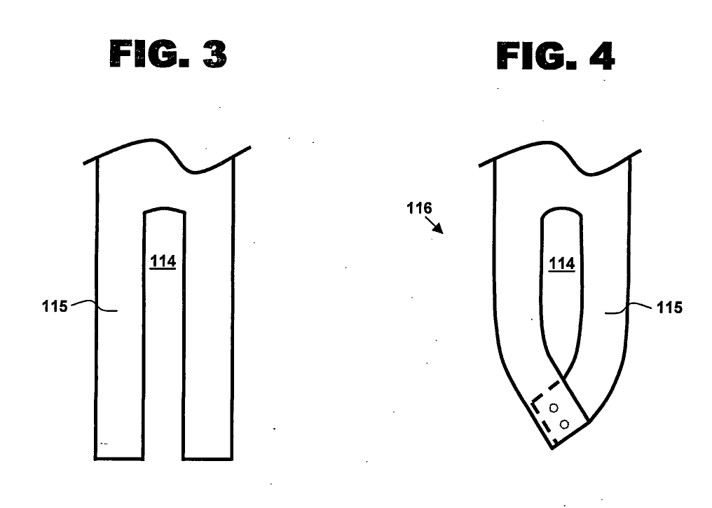

In the present embodiment, each leg 112 includes a snap-acting spring tip 116 at the free end of the leg (i.e., the end not attached to body 111). To create a snap-acting spring tip 116, a longitudinal slot 114 is formed in the free end of leg 112, resulting in two tip segments 115, as best seen in FIG. 3. The shape of slot 114 may vary from that shown. Tip segments 115 of each individual leg are overlapped and affixed one to another by, for example, spot welding, as is seen in FIG. 4. Snap-acting spring tip 116 thus formed is distorted out-of-plane with respect to the adjacent portion of leg 112. This out-of-plane distortion allows snap-acting spring tip 116 to function as a bistable snap-acting element, as described in further detail below.

Spicule 117, best seen in FIG. 1, is attached to the free end of each leg 112 to aid snap-acting spring tip 116 in piercing into the tissue of the valve annulus. Spicule 117 may surround, be included in, or otherwise be affixed to the joint forming snap-acting spring tip 116. To increase the ability of legs 112 to grip the tissue of the valve annulus, at least one barb 118 is cut into each leg, with the sharp end of the barb pointing towards body 111 as seen in FIGS. 1, 2, 5 and 6. Pointing in this direction, the barb does not interfere with leg 112 entering the tissue of the valve annulus but barb 118 engages with the tissue when leg 112 reverses direction, thus preventing the leg from pulling back out of the valve annulus once it has entered the tissue. One skilled in the art will recognize that other shapes and orientations of barbs may be used to secure legs 112 within the tissue of a valve annulus.

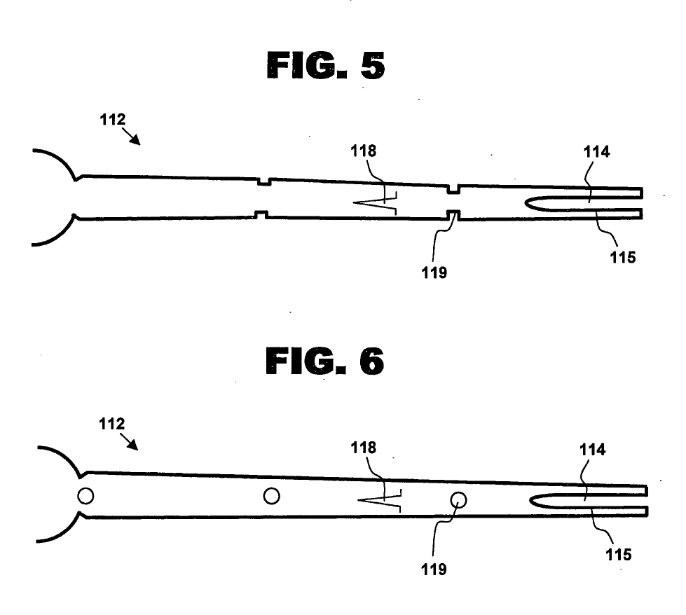

Each leg includes one or more deformation elements 119 designed to produce localized weakened areas in the leg. As will be explained more fully below, these weakened areas act as “knee” portions, allowing the legs to plastically bend in these areas. As seen in FIGS. 1 and 2, the deformation elements are notches, which are formed substantially opposite one another on the edges of legs 112. In another embodiment, deformation elements 119 may be, for example, corrugations or perforations (shown in FIGS. 5 and 6, respectively) or other structures known in the art that are capable of locally weakening legs 112.

During manufacture, legs 112 extend from body 111 and are heat set or otherwise preformed such that each leg 112 tends to self-deploy radially outward from the longitudinal axis of body 111 when contracting device 110 is released from a delivery catheter. FIG. 1 shows legs 112 in their splayed, preformed configuration. FIGS. 5 and 6 show legs prior to being preformed.

Contracting device 110 is a bistable apparatus having a first stable deployed state as shown in FIG. 1, where contracting device 110 has been released from a delivery catheter to assume the configuration into which legs 112 were preformed during manufacture. The device is capable of transitioning to a second stable treatment state as shown in FIG. 2. Transition of legs 112 and snap-acting spring tips 116 from their deployed states to their treatment states is accomplished by applying axial force to body 111.

When axial force is applied to body 111, snap-acting spring tips 116, with spicules 117, are pressed against the surface of the valve annulus and begin to pierce into the tissue of the annulus. At a certain threshold force, snap-acting spring tips 116 will transform “over-center” from their deployed state generally perpendicular to the plane of the valve annulus into their treatment state wherein they are directed generally radially inward toward the longitudinal axis of body 111. In addition, in response to applying the axial force, one or more weakened areas of legs 112 (the “knees” resulting from deformation elements 119) permanently bend, deforming the device into its contracting, treatment state, as shown in FIG. 2. Deformation elements 119 thus act as mechanical “fuses,” that respond to the axial force by “failing,” thereby creating local deformations. Contracting device 110 transitions to its stable treatment state as a combination of the transformation of snap-acting spring tips 116 and the permanent bending of deformation elements 119. In this stable treatment state, legs 112 of contracting device 110 draw the valve annulus toward its center.

In another embodiment, the entire device may be composed of a shape memory metal alloy that will achieve the desired mechanical profile (the treatment state) when the device is released from the delivery catheter.

It is desirable that contracting device 110 be visible using fluoroscopy, echocardiography, intravascular ultrasound, angioscopy, or another means of visualization to aid in positioning. Where fluoroscopy is utilized, any or all of contracting device 110 may be coated with a radiopaque material, or a radiopaque marker may be included on any portion of the device that would be useful to visualize.

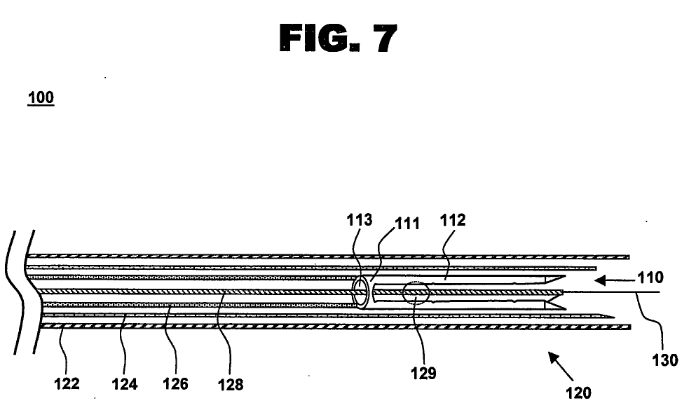

Another aspect of the present invention is a system for contracting tissue in a mammalian body that includes contracting device 110 described above. System 100 is shown in FIGS. 7-13, and further includes delivery catheter 120 and guidewire 130. Only a distal portion of the system is illustrated. The terms “distal” and “proximal” are used herein with reference to the treating clinician during deployment of the device; “Distal” indicates a portion distant from, or a direction away from the clinician and “proximal” indicates a portion near to, or direction towards the clinician.

System 100 is described below in the context of radially contracting a mitral valve annulus to effect a mitral valve repair. However, it may also be used to reduce the compass of other openings and structures within the body.

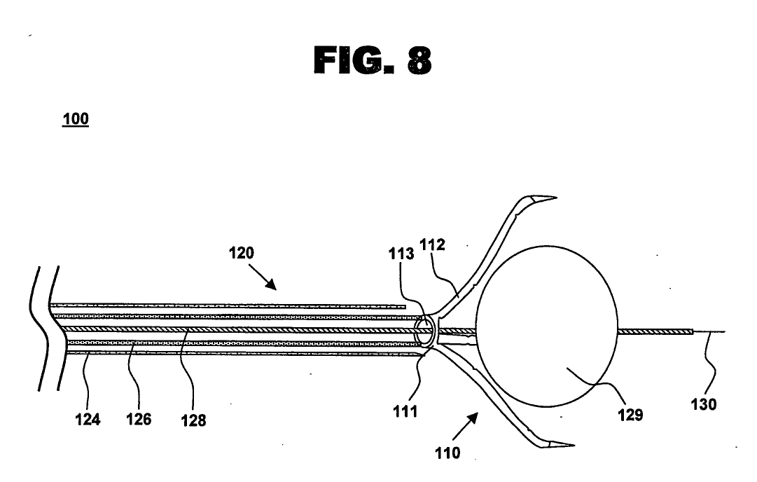

Besides the two stable deployment and treatment states discussed above, contracting device 110 may be deformed into a radially compressed configuration when confined within catheter 120 for delivery, as shown in FIG. 7. Contracting device 110 is capable of self-expansion from the radially compressed delivery configuration to the first stable deployment configuration, as shown in FIG. 8. The compression of contracting device 110 into the radially compressed configuration may be achieved elastically, that is, without any permanent deformation of the device.

In the present embodiment, delivery catheter 120 comprises guiding sheath 122, holding tube 124, push tube 126, and balloon catheter 128. Holding tube 124 is slidable within a lumen of guiding sheath 122, push tube 126 is slidable within a lumen of holding tube 124, and balloon catheter 128 is slidable within a lumen of push tube 126. At least a portion of balloon catheter 128 is additionally slidable within aperture 113 of contracting device 110. Thus, delivery catheter 120 comprises four separate telescoping members, each slidable to be individually extended or retracted as needed to deliver contracting device 110.

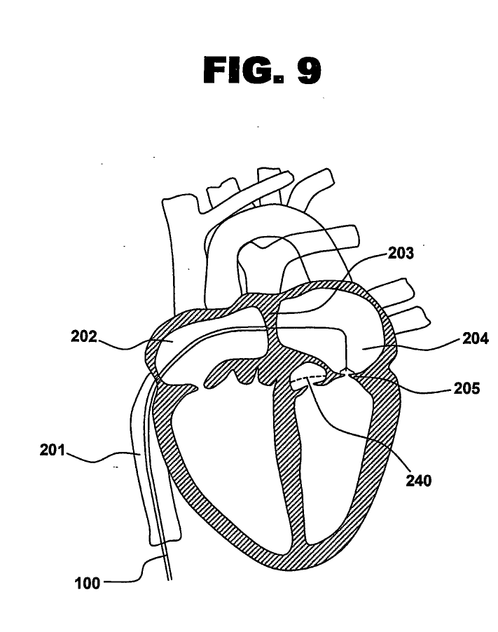

Guiding sheath 122 comprises a flexible, biocompatible material such as polyurethane, polyethylene, nylon, or polytetrafluoroethylene (PTFE). Guiding sheath 122 has a preformed or steerable distal tip that is capable of assuming a desired bend with respect to the longitudinal axis of the sheath to aid in delivering the system. In one embodiment, this bend allows system 100 to approach the interatrial septum at the correct orientation to deliver contracting device 110 through the septum as seen in FIG. 9. In the illustrated embodiment, system 100 is passed through inferior vena cava 201 into right atrium 202, then guiding sheath 122 remains within the right atrium while the holding tube and its contents pierce through interatrial septum 203 (also referred to below as the septal wall) into left atrium 204 to be positioned adjacent to mitral valve 205. Those skilled in the art will appreciate that alternative paths are available to gain access to the mitral valve.

Holding tube 124 comprises the same or a different biocompatible material from that used to form guiding sheath 122. Like guiding sheath 122, holding tube 124 has a preformed or steerable distal tip that is capable of assuming a desired bend with respect to the longitudinal axis of the tube when the tube is extended beyond guiding sheath 122. Where the tip is preformed, the biocompatible material comprising holding tube 124 must allow the distal tip to assume a linear configuration while contained within the guiding sheath and the tip will assume the desired, preformed bend when extended beyond the distal end of guiding sheath 122. In the embodiment shown in FIG. 9, the bend allows system 100 to be directed toward mitral valve 205.

In the present embodiment, the distal end of holding tube 124 is angle-cut to form a sharp edge able to pierce through interatrial septum 203. Thus, where contracting device 110 Is to be delivered transluminally, holding tube 124 must be flexible enough to be delivered through vasculature to the treatment area while still rigid enough to pierce the septal wall.

Push tube 126 also comprises a biocompatible material. Push tube 126 must be axially flexible for transluminal delivery while being longitudinally incompressible to exert an axial force on body 111 of contracting device 110, as described below.

In the present embodiment, balloon catheter 128 includes a single low-pressure balloon 129. During delivery, balloon 129 is positioned between legs 112 of contracting device 110 as shown in FIG. 7. The balloon may be partially inflated to a diameter greater than the diameter of aperture 113 in the body of contracting device 110, thereby serving as a retaining device for contracting device 110 during delivery.

In FIG. 8, contracting device 110 is shown released from delivery catheter 120 with legs 112 self-expanded, the device having assumed its stable deployed state. In the present embodiment, push tube 126 propels contracting device 110 out of holding tube 124, at which time legs 112 self-expand or splay away from the longitudinal axis of body 111. Alternatively, contracting device 110 may be released by retracting holding tube 124 while maintaining contracting device 110 stationary with push tube 126. Guiding sheath 122 is not seen in FIG. 8 as it remains in right atrium 202, supporting the delivery system while holding tube 124 and its contents are advanced through septal wall 203 and into position adjacent to mitral valve 205.

As shown in FIGS. 8 and 10, balloon catheter 128 and push tube 126 may be extended from holding tube 124 simultaneously to maintain balloon 129 in a position distal to body 111 and substantially within legs 112. In the present embodiment, balloon catheter 128 is directed over guidewire 130, which is passed through mitral valve 205 prior to extending balloon catheter 128. Balloon 129 is expanded at approximately the same time contracting device 110 is released from holding tube 124. As seen in FIG. 10, balloon 129 is positioned over and partially within mitral valve 205. Balloon 129 thus acts as both a retaining device and a positioning device to ensure proper placement of contracting device 110 over the valve annulus.

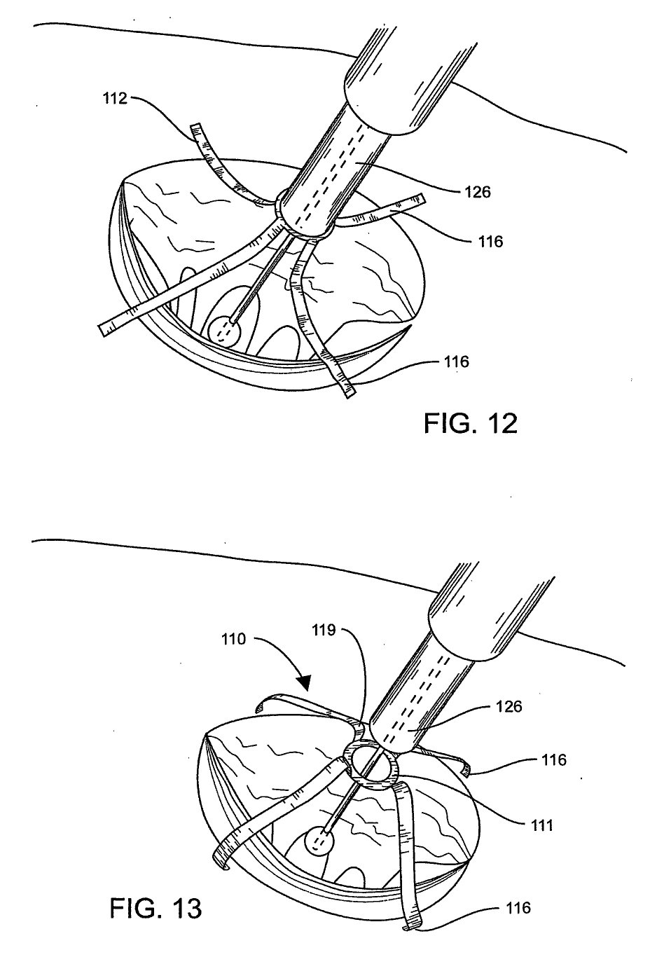

As shown in FIG. 11, balloon 129 is then at least partially deflated. At the same time, push tube 126 exerts an axial force on body 111 to drive contracting device 110 into contact with the mitral valve annulus (FIG. 12). As push tube 126, now acting as a compression device, continues to exert an axial force on body 111, snap-acting spring tips 116 transform “over-center” and deformation elements 119 bend locally (FIG. 13) to transition contracting device 110 to its treatment state having an inherent, stable contraction force directed radially inward toward the longitudinal axis of body 111.

Once contracting device 110 has assumed its treatment state, thereby contracting the valve annulus and effecting a mitral valve repair, balloon 129 may be deflated and withdrawn through aperture 113, allowing delivery catheter 120 to be removed from the body.

One skilled in the art will appreciate that numerous other embodiments of the system are possible, and that such embodiments are contemplated and fall within the scope of the presently claimed invention. For example, the system described above may further include a gripping device, for example biopsy forceps, for holding the contracting device until it is properly positioned upon the valve annulus. Alternatively, biopsy forceps or another gripping device may replace both the balloon catheter and the push tube, holding the contracting device for positioning and also applying an axial force to the device, thus acting as both a positioning device and a compression device. In another alternative, the balloon catheter may be eliminated, with a push tube deploying the contracting device and a retaining device positioning the contracting device. In yet another alternative, the balloon catheter may include two balloons, one initially positioned proximal to aperture 113 and the other positioned distal to the aperture. The distal balloon is a low pressure balloon as described above, and the proximal balloon is a high pressure balloon capable both of pushing the contracting device out of the delivery catheter and of exerting an axial force on the contracting device. Still another alternative includes a magnetic guidewire positioned within the coronary sinus prior to deployment of the device in the atrium. For example, guidewire 240, shown in phantom in FIG. 9 may be magnetic. In this embodiment, the legs of the contracting device are magnetic. Upon deployment of the contracting device within the atrium, the magnetic guidewire acts as a positioning device to attract the contracting device, causing the contracting device to be properly drawn into position.

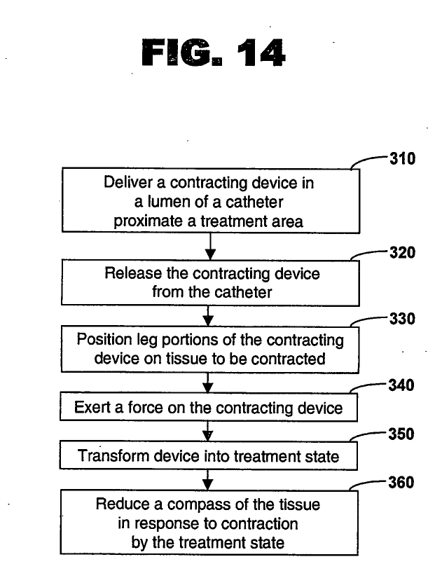

Another aspect of the present invention is a method of contracting tissue in a mammalian body. FIG. 14 shows a flow diagram of one embodiment of the method in accordance with the present invention.

A contracting device is delivered in a lumen of a catheter proximate a treatment area (Block 310). In the present embodiment, the contracting device and catheter are those comprising system 100, as described above.

For delivery, system 100 is in the configuration shown in FIG. 7, with contracting device 110 slidably received within delivery catheter 120. Delivery catheter 120 carrying contracting device 110 is passed through the venous system and into a patients right atrium adjacent to the mitral valve. This may be accomplished as shown in FIG. 9, in which delivery catheter 120 has been inserted through the femoral vein into the common iliac vein, through inferior vena cava 201 into right atrium 202, and then at least a portion of the delivery catheter is passed through septal wall 203 into left atrium 204 and positioned adjacent to mitral valve 205.

Other paths are available, including through the radial vein into the brachial vein, through the subdlavian vein, through the superior vena cava into the right atrium, and then transeptally into the left atrium. Yet another possible path would be through the femoral artery into the aorta, through the aortic valve into the left ventricle, and then through the mitral valve into the left atrium. Still another possible path would be through the left or right pulmonary vein directly into the left atrium. For surgical approaches with an open chest, the delivery catheter may be replaced by an elongate element such as an endoscope, or a trocar or cannula inserted directly into the superior vena cava or the aortic arch. The elongate element can then follow the same path as the catheter-based procedure to reach the left atrium, either transeptally or through the cardiac valves. Transeptal approaches, whether percutaneous or surgical, may require placement of a closure device at the transeptal puncture on removal of the catheter or other elongate element after the procedure.

The contracting device is released from the catheter (Block 320). In the present embodiment, this is accomplished by extending push tube 126 to propel contracting device 110 out of holding catheter 124, as is seen in FIG. 8.

Legs of the contracting device are positioned on tissue to be contracted (Block 330). In the present embodiment, the legs are positioned on a mitral valve annulus. As described in detail above and shown in FIG. 10, balloon 129 of balloon catheter 128 is positioned partially within mitral valve 205, thereby positioning legs 112 of contracting device 110 on the valve annulus.

A force is exerted on the contracting device (Block 340). In the present embodiment, this is accomplished by push tube 126 exerting an axial force on body 111 of contracting device 110. The tips of legs 112 are embedded within the tissue of the valve annulus, and contracting device 110 is transformed into a treatment state (Block 350) by “over-center” action of snap-acting spring tips 116 and by permanently bending deformation elements 119 to form “knees” in legs 112. A compass of the tissue, in the present embodiment the diameter of the valve annulus, is thus reduced in response to the contracting device 110 attaining its contracted, treatment state (Block 360). Those of skill in the art will recognize that the phrase “diameter of the valve annulus” is used for simplicity in teaching the invention; A mitral valve is not exactly circular, being more D-shaped. Thus, it will be understood that, in the treatment state, each leg 112 of contracting device 110 may shorten a corresponding radial dimension of tissue engaged by the device.

While the embodiments of the invention disclosed herein are presently considered to be preferred, various changes and modifications can be made without departing from the spirit and scope of the invention. The scope of the invention is indicated in the appended claims, and all changes and modifications that come within the meaning and range of equivalents are intended to be embraced therein.

What is claimed is:

1. A device for contracting tissue in a mammalian body, comprising:
a body having a longitudinal axis; and
a plurality of legs arranged about the body, each leg having one end coupled to the body, the plurality of legs being radially splayed about the axis, each leg including a snap-acting spring tip engageable with the tissue, each leg being capable of transformation between a deployment state and a treatment state.

2. The device of claim 1 wherein the spring tips are closer to the longitudinal axis when the legs are in the treatment state than the tips are when the legs are in the deployment state.

3. The device of claim 1 wherein each snap-acting spring tip comprises two tip segments affixed one to another.

4. The device of claim 1 further comprising:
a spicule attached to each spring tip.

5. The device of claim 1 wherein each leg further includes at least one deformation element capable of forming a localized bend in response to an axial force applied to the leg.

6. The device of claim 1 wherein the at least one deformation element is selected from a group consisting of a notch, a perforation, a corrugation, and a combination thereof.

7. The device of claim 1 wherein each leg further includes at least one barb directed towards the body.

8. The device of claim 1 wherein at least the legs comprise a material selected from a group consisting of a nickel-titanium alloy, a nickel-cobalt alloy, a cobalt alloy, a thermoset plastic, stainless steel, a stainless steel alloy, a biocompatible shape-memory material, a biocompatible superelastic material, and a combination thereof.

9. The device of claim 1 wherein at least a portion of the device includes a therapeutic agent selected from a group consisting of an antithrombotic, an anticoagulant, an antibiotic, an anti-inflammatory, and a combination thereof.

10. The device of claim 1 wherein at least a portion of the device is radiopaque.

11. The device of claim 1 wherein at least one radial dimension of a mitral valve annulus is shortened when the legs are in the treatment state.

12. A system for contracting tissue in a mammalian body including the contracting device of claim 1 and further comprising:
a delivery catheter, wherein the contracting device is slidably received within a lumen of the delivery catheter.

13. The system of claim 12 further comprising:
a guidewire slidably received within a lumen of the delivery catheter.

14. The system of claim 12 further comprising:
a magnetic guidewire for positioning within a coronary sinus.

15. The system of claim 12 wherein the delivery catheter comprises a compression device.

16. The system of claim 12 wherein the delivery catheter comprises a positioning device.

17. The system of claim 12 wherein the delivery catheter comprises a guiding sheath, a holding tube slidably received within a lumen of the guiding sheath, a push tube slidably received within a lumen of the holding tube, and a balloon catheter including at least one balloon and being slidably received within a lumen of the push tube.

18. The system of claim 17 wherein the push tube acts as a compression device.

19. The system of claim 17 wherein the at least one balloon acts as a positioning device.

20. The system of claim 17 wherein the at least one balloon acts as a compression device.

21. The system of claim 12 wherein the legs are in a radially compressed configuration while the contracting device is within a lumen of the delivery catheter and wherein the legs self-expand when the contracting device is released from the delivery catheter.

22. A method of contracting tissue in a mammalian body, comprising:
delivering a contracting device in a lumen of a catheter proximate a treatment area;
releasing the contracting device from the catheter;
positioning legs of the contracting device on tissue to be contracted;
exerting a force on the contracting device;
transforming the device into a treatment state; and
reducing a compass of the tissue in response to the treatment state.

23. The method of claim 22 further comprising:
bending the legs in response to the force.

24. The method of claim 22 wherein reducing a compass of the tissue in response to the treatment state comprises reducing a diameter of a mitral valve annulus to effect a mitral valve repair in response to the treatment state.
